(19) United States
(12) Patent Application Publication
(10) Pub. No.: US 2025/0190621 A1
(43) Pub. Date: Jun. 12, 2025

(54) SELECTIVELY DE-IDENTIFYING DATA

(71) Applicant: BECKMAN COULTER, INC., Brea, CA (US)

(72) Inventors: Kasiviswanathan BOOMINATHAN, Bangalore (IN); Chetan SESHADRI, Bangalore (IN); Bhagaban NAYAK, Chaska, MN (US); Steven THACHER, St. Louis Park, MN (US)

(21) Appl. No.: 19/057,912

(22) Filed: Feb. 19, 2025

Related U.S. Application Data
(63) Continuation of application No. PCT/IB2023/058179, filed on Aug. 14, 2023.
(60) Provisional application No. 63/399,520, filed on Aug. 19, 2022.

(51) Int. Cl. G06F 21/62; G16H 10/60

(52) U.S. Cl. CPC G06F 21/6254; G16H 10/60

(57) ABSTRACT

A computer implemented method, a computing device, a laboratory instrument, a computer program product and a computer readable storage medium for selectively de-identifying protected health information (PHI) are provided. The method comprises accessing a first data file, the first data file comprising at least a first data item, wherein the first data item comprises PHI and first PHI category information, wherein the first PHI category information is indicative of a first PHI category of a plurality of PHI categories, the PHI in the first data item belonging to the first PHI category. The method further comprises accessing the first PHI category information. The method further comprises assessing, based on the first PHI category information, whether the PHI in the first data item is to be de-identified or not. If the PHI in the first data item is to be de-identified, the method further comprises generating a second data item by modifying the first data item such that the protected health information is de-identified.

### CROSS REFERENCE TO RELATED APPLICATIONS

This is a continuation of International Pat. App. No. PCT/IB2023/058179, entitled “Selectively De-identifying Data”, filed Aug. 14, 2023 and published as WO2024/038370 on Feb. 22, 2024, while claims priority to U.S. Provisional Application 63/399,520, entitled “Selectively De-identifying Data,” filed Aug. 19, 2022, the disclosures of which are incorporated by reference herein.

### TECHNICAL FIELD

The disclosed technology relates to handling health information that can be linked to a specific individual, i.e., protected health information.

### BACKGROUND

A laboratory instrument (e.g., a chemistry analyzer) may analyze or carry out a clinical test on a biological sample (or sample), such as serum, urine, plasma. Analysis data may be generated based on the analysis. The analysis data may include information that can be linked to a specific individual. Such information may be referred to as protected health information.

It may be desirable to transmit analysis data including protected health information over a computer network, possibly by transmitting the analysis data from an internal network to an external entity. Operations may be performed on the protected health information prior to transmission in view of the sensitivity of protected health information.

### SUMMARY

It is an object of some embodiments to improve the usability of analysis data for troubleshooting, testing or performance analysis. Specifically, it is an object of the some embodiments to improve the usability of analysis data for troubleshooting, testing (e.g., a laboratory instrument) or performance analysis in a laboratory environment.

This object is achieved as set out in the independent claims. Further implementations are the subject matter of the dependent claims.

According to a first aspect, a computer implemented method for selectively de-identifying protected health information (PHI) is provided. The method comprises accessing a first data file, the first data file comprising at least a first data item, wherein the first data item comprises PHI and first PHI category information, wherein the first PHI category information is indicative of a first PHI category of a plurality of PHI categories, the PHI in the first data item belonging to the first PHI category. The method further comprises accessing the first PHI category information. The method further comprises assessing, based on the first PHI category information, whether the PHI in the first data item is to be de-identified or not. If the PHI in the first data item is to be de-identified, the method further comprises generating a second data item by modifying the first data item such that the protected health information is de-identified. The method may be carried out by one or more computing devices. A computing device may comprise at least one memory and at least one processor. A computing device may also comprise one or more input/output units.

Accessing the first data file may comprise retrieving the first data file from the at least one memory of the computing device or from remote data storage (e.g., a database, secondary memory, cloud storage or the like). Accordingly, in some cases, accessing the first data file may comprise downloading data. For example, the computing device may retrieve the data file in response to a user command.

Additionally or alternatively, accessing the first data file may comprise receiving the first data file, e.g., from a user or another computing device. For example, the computing device may receive the first data file from another computing device via a computer network. Specifically, the first data file may be received from a laboratory instrument. The laboratory instrument may be an analyser, i.e., an apparatus configured to carry out one or more clinical tests on a biological sample.

More specifically, the laboratory instrument may be a chemistry or hematology analyzer. The two options for accessing the first data file are not mutually exclusive. For example, accessing the first data file may include receiving the first data file, during the first data file in the memory of the computing device and retrieving the first data file by accessing the memory.

The first data file includes at least the first data item. The first data file may include a plurality of entries. Each entry may include one or more data fields (hereinafter also referred to as “fields”) or components. Each field may include one or more data items. Each entry in the first data file may have a corresponding structure (e.g., the same number of fields, fields having the same data type). Unless otherwise indicated, information provided below with regard to the first data item may also be applicable to the other data items in the first data file.

The first data file may be a text file, i.e., a computer file that is structured as a sequence of lines of electronic text. The text file may be substantially human readable. The text file may be distinguished from a binary file by an operating system (e.g., Microsoft Windows or Linux) of the computing device. The first data file may be formatted. The first data file may include plaintext data items or data items in a log format such as JavaScript Object Notation (JSON), Windows event log, Common Event Format (CEF) or the like. The first data item may comprise descriptive markup language (such as LaTeX, HTML, XML, and the like).

Data items in the first data file may be structured and/or encoded. For example, the first data file may separate data using multiple delimiters. A first delimiter (e.g., a comma or a vertical line) may be used to separate fields and a second delimiter (e.g., a newline) may be used to separate entries within the first data file. A data item in the first data file may be part of a field including other data items or may encompass an entire field.

Protected health information (PHI) may be information that has been identified as sensitive and needing protection. For example, a subset of information output by a laboratory instrument may be identified as PHI. In the context of PHI, the term “protected” indicates that the health information is sensitive and should be protected and does not necessarily indicate that the health information has already been protected.

PHI may refer to information that can be used to identify a specific individual. Such identification information may include a direct identifier, such as a name or government identification number or an indirect identifier, such as a birthday, city of birth, age, gender, or the like. A direct identifier may be usable to uniquely identify an individual. An indirect identifier might not be unique to an individual but may be usable in combination with other indirect identifiers to uniquely identify the individual. Further indirect identifiers are, for instance, the name of the physician that ordered the test or the name of the attending physician. Accordingly, PHI may include one or more direct identifiers and/or one or more indirect identifiers.

A PHI category (e.g., the first PHI category) may be a class or division of PHI having shared characteristics. More specifically, the PHI category may group fields corresponding to a single direct identifier or fields corresponding to a single indirect identifier. For example, a sample ID category may group PHI identifying a biological sample; PHI in the sample ID category may be preceded by specified text or prefix (e.g., sample ID:) and/or appear in a specified location of an entry in the first data file (e.g., in the fifth field along with descriptive text). Sample ID may be an indirect identifier. Other PHI categories may include Patient ID category, DoctorlD category or the like. There may be any number of PHI categories. For example, there are two PHI categories, e.g. the SamplelD category and the Default Category, the default category being the category of PHI that does not belong to the Sample ID category. For instance, there are three PHI categories, e.g. the SamplelD category, the Patient ID category and the Default Category, the default category being the category of PHI that does not belong to the Sample ID category and the PatientID category. In particular, there are four PHI categories, e.g. the SamplelD category, the Patient ID category, the DoctorlD category and the Default Category, the default category being the category of PHI that does not belong to the Sample ID category, to the PatientID category and to the DoctorlD category. The PHI category may also correspond to a subset of a group of PHI (e.g., selected sample IDs being tested).

Each of the PHI categories may be unique. PHI category information (e.g., first PHI category information) may indicate a PHI category (e.g., the first PHI category). For example, the PHI category information may distinguish data items' PHI belonging to the PHI category from PHI of other data items that does not belong to the PHI category. PHI category information, e.g. the first PHI category information, of a data item, e.g. of the first data item, may be comprised in a string of characters detectable by a parser, e.g., via one or more regular expressions (i.e., pattern matching). The PHI category information may be provided by a prefix and/or a suffix of the data item. For example, the aforementioned prefix and the suffix may appear at the beginning and at the end of the data item such that the PHI of the first data item is contained within the category information. The prefix and the suffix may be the same. Alternatively, the prefix may differ from the suffix. The prefix and the suffix may comprise alphanumeric characters (a, b, c, 1, 2, 3, and the like) and/or nonalphanumeric characters (*, >, >, $, &, %, I, and the like). Alternatively, the PHI category information may be comprised in a string which is either prepended or appended to the string comprising the PHI.

The PHI category information may include a combination of characters that do not otherwise appear (i.e., are not present elsewhere) in the first data file. For example, the prefix and/or the suffix may each include the combination of characters. The combination of characters may include alphanumeric and/or non-alphanumeric characters. Alternatively, the combination of characters may be limited to non-alphanumeric characters or to word characters (alphanumeric characters as well as the underscore character).

PHI category information, e.g. the first PHI category information, of a data item, e.g. of the first data item, may be comprised in an XML element marked by a PHIcategory-dependent tag. In particular, the PHI may be the content of the XML element. PHI category information, e.g. the first PHI category information, of a data item, e.g. of the first data item, may be comprised in a PHI category-dependent LaTeX command. The PHI may be the parameter of the command. For example, each PHI category of the plurality of PHI categories may be associated to a respective color. If this is the case, the PHI category-dependent LaTeX command may be a command specifying the text color of its parameter. If this is the case, in the first data item, PHI belonging to a PHI category is the parameter of the command specifying as text color the color associated to said PHI category as text color.

In addition to PHI, the entry in the first data file may include unprotected analysis data (i.e., unprotected data). The unprotected analysis data may include one or more timestamps (i.e., a sequence of characters or encoded information identifying when an event occurred, possibly including a date and time of day or being a number of seconds relative to a time in the past). For example, the timestamps may include the start and end of an event corresponding to the first data item (e.g., the first data item may include information regarding the event).

In addition, the unprotected data may include an entry identifier, a severity level and a description of the event, the description of the event may include a rack position and/or a rack identifier in addition to descriptive text. The rack position and the rack identifier may refer to the laboratory instrument, which may comprise one or more racks. The racks may be suitable for holding sample cups or test tubes. The racks may be placed on rack input trays. For example, there may be two rack input trays, each holding a maximum of twenty racks, for a maximum of 200 samples. The unprotected analysis data may also include an error message and/or additional information for analyzing the event.

The PHI in the first data item belongs to the first PHI category. The first data item may be limited to PHI in the first PHI category and might not include PHI in any other PHI category.

Accessing the first PHI category information may include using one or more specified regular expressions to scan the first data item and/or the first data file. The accessing of the first PHI category information may be implemented along with accessing the first data file (e.g., as part of the same program or function call) or may be implemented separately. A regular expression may include a sequence of characters (e.g., metacharacters and regular characters) specifying a search pattern to describe and/or parse text. Accordingly, the specified regular expressions may be used to isolate or determine the first PHI category information in the first data item.

Assessing, based on the first PHI category information, whether the PHI in the first data item is to be de-identified or not may include using the first PHI category information to determine whether the PHI in the first data item is to be de-identified. Accordingly, a category setting may be associated with the first PHI category information. If the category setting is turned off (i.e., set to false), then the first data item might be de-identified. Similarly, if no category setting is associated with the first PHI category information, then the first data item might be de-identified. Alternatively, if the category setting is turned on (i.e., set to true), then the first data item might be handled differently (i.e., distinctly) from other PHI. For example, when the category setting is turned on, the first data item might not be de-identified. Alternatively, when the category setting is turned on, the first data item might be de-identified in such a way that it is possible to reconstruct the original data after de-identification. For example, when the category setting is turned on, the first data item might be encrypted.

An advantage of including PHI category information in a data item (and thereby in a data file), as opposed to a configuration file or another external file external to the data file, is that the PHI category information can be more easily identified, e.g., with regular expressions. If the PHI category information were not in the data item, then the external file would need to include detailed instructions about where to find PHI information, e.g., a specified field number in a log file entry as well as a location within the field and a format (e.g., character classes included in the PHI, such as alphanumeric or printable characters or blanks). By including the PHI category information in the data item, it is not necessary to provide such detailed instructions in the external file and the PHI information can be more efficiently located.

De-identification of information (e.g., PHI) may prevent or hinder the identity of an individual associated with the PHI from being revealed, i.e., hinder or prevent re-identification. Hence, after de-identification of PHI, it may be impossible or very difficult to identify an individual from the de-identified PHI. De-identifying the first data item may include generating the second data item, wherein the PHI may be replaced or obfuscated.

For example, the PHI of the first data item may be replaced with a specified (e.g., predefined) string in the second data item, such that the specified string cannot be used to reconstruct the original PHI. Accordingly, PHI having the Patient Name category in the first data item may be replaced with the specified string including four asterisks, i.e. the string “****” in the second data item. In the example, since all data items in the category are replaced with the same specified string, the second data item cannot be used as a basis for arriving at the first data item; hence, the replacement is irreversible. The specified string may include alphanumeric and/or nonalphanumeric characters. Alternatively, the specified string may be limited to nonalphanumeric characters.

Alternatively, generating the second data item may include modifying the first data item by encrypting the PHI of the first data item. As yet another alternative, the PHI in the first data item may be replaced with a code that can be decoded by a third party (e.g., a doctor) possessing a key. For example, a patient name may be replaced with a corresponding coded name that can be decoded by the third party using a table matching patient names to corresponding coded names. In the preceding two examples, the replacement is reversible, i.e., the second data item can be used as a basis for arriving at the first data item.

De-identification may be handled differently depending on a destination of the second data item. For example, the second data item may be exported to the GUI or to a second data file. When exported to the GUI, if a user does not have permission to view the PHI, the PHI may be de-identified (e.g., obfuscated) by using a series of symbols, e.g., asterisks. If the user does have permission to view the PHI, then the PHI may be shown as readable text in the GUI. When exported to the second data file, the PHI may be de-identified by replacing the PHI with a specified series of characters. Conventionally, all PHI in a data file is de-identified (e.g., irreversibly obfuscated) before export of data in the data file, e.g., to a GUI or to another data file (where the other data file may be transmitted to an external entity). The de-identification may be useful in order to protect sensitive data.

De-identification may be enforced at compile time by encapsulating PHI information within a specified class before the PHI information is displayed or logged. A PHI handling framework may enforce de-identification using a code analyzer (e.g., the .NET Compiler Platform, also referred to as Roslyn). A compile time error may be thrown if it is determined that PHI has not been encapsulated for de-identification. The encapsulation may include mapping and marking the PHI in a class of the PHI framework. However, it may be desirable to prevent de-identification or irreversible de-identification of selected PHI.

Accordingly, protected health information is selectively de-identified by generating the second data item. The selective de-identification is carried out by means of the first PHI category information, which is a basis for determining whether the PHI in the first data item is to be de-identified. De-identification may be carried out based on the risk of whether the first data item can be used to arrive at the identity of the individual associated with the PHI. In addition, the de-identification may be carried out based on whether the first data item may be useful for troubleshooting or testing, e.g., troubleshooting or testing a laboratory instrument.

Selective de-identification ensures that the risk that a data item can be used to arrive at the identity of the individual associated with the PHI is low, while also facilitating troubleshooting or testing. For example, if the risk that the first data item can be used to arrive at the identity of the individual associated with the PHI is relatively low and the first data item appears to be useful for troubleshooting then the category setting may be associated with the first data item (e.g., the first data item might not be modified) and the second data item might not be generated. Relatively low risk of reidentification may be associated with sample or laboratory equipment information, such as a samplelD or a TubeID associated with the samplelD. Sample or laboratory equipment information may also be relatively useful for troubleshooting or testing. Conversely, relatively high risk of reidentification may be associated with patient information, such as patient gender and birth date. Patient information is also unlikely to be useful for troubleshooting or testing.

Troubleshooting may refer to determining the cause of an event (e.g., an error or problem) and may further include determining how to prevent the event from happening again. For example, troubleshooting a laboratory instrument may include determining what happened to (or what went wrong with) an order from a physician or a result from a test. Troubleshooting may include traceability, e.g., determining all the entities involved in causing the event, and/or tracking, e.g., following movement of an object associated with the event.

Traceability may refer to the determination of entities (e.g., laboratory instruments) involved in the event. For example, laboratory instruments A and B may contribute to an error at laboratory instrument C. A data item such as the sample ID may be useful for tracing the error at laboratory instrument C back to laboratory instruments A and B. Tracking may refer to following movement of an object (e.g., a sample) to different locations (e.g., two different laboratory instruments). For example, a sample may be analyzed by multiple laboratory instruments during the course of a diagnostic process. Tracking may be useful during troubleshooting, e.g., to determine where something went wrong in the course of analysis, or during performance analysis (e.g., to determine whether a sample was optimally routed through the laboratory instruments e.g. of an automated laboratory instrument).

Testing may refer to determining whether a laboratory instrument is functioning properly.

Information that is not selectively de-identified may also be used for performance analysis, e.g., developing statistics regarding laboratory instruments (e.g., minutes of idle time per day/week, number of samples processed, number of samples processed per physician). For example, the samplelD may be used in conjunction with one or more log entry timestamps to track whether the samples have been timely processed, e.g. each sample has been processed within its stability period. In particular, the stability period of a sample is the amount of time after the drawing of the sample in which the sample may be reliably analysed by the laboratory instruments. Alternatively, the samplelD may be used to determine the number of samples per patient processed. For instance, such metric may be useful for determining whether the aliquotation of primary samples is carried out as expected.

An example of the use of selective de-identification in the context of testing follows. The first PHI category may correspond to a test category. The first PHI category may be used when the automated laboratory system, or a laboratory instrument of the automated laboratory system, is being tested. Specifically, the automated laboratory system may include a flow cytometer for virus detection (i.e., a flow cytometer used for virometry). The flow cytometer may be capable of processing 32 sample tubes. When testing, two of the sample tubes may contain known control samples, e.g., a sample known to contain a virus and a sample known to be free of the virus. It may be unknown whether the other samples 30 samples (i.e., experimental samples) contain the virus. In the example, the PHI information for the sample IDs of the control samples is not de-identified in order to be able to track the control samples during the test. In contrast, the PHI information for the sample IDs of the experimental samples may be de-identified.

In this way, by means of the first PHI category, sample IDs of the control samples are not de-identified; accordingly, the control samples can be used for testing functionality of the flow cytometer and also discarded for subsequent analysis, assuming that results of the testing show that the flow cytometer is working properly. In contrast, sample IDs of the experimental samples are de-identified, along with other PHI information, since the experimental samples are not used for testing the flow cytometer.

The method may further comprise accessing PHI-presence information in the first data file, the PHI-presence information being indicative of whether the first data item comprises protected health information, and assessing, based on the PHI-presence information, whether the first data item comprises protected health information or not.

For example, accessing the PHI presence information in the first data file may include accessing the PHI presence information in the first data item. Accordingly, PHI presence information for the first data item may be entirely contained within the first data item.

The PHI presence information may be comprised in a string of characters detectable by a parser, e.g., via one or more regular expressions (i.e., pattern matching). The PHI presence information may be provided by a prefix and/or a suffix of the first data item. The prefix and the suffix may be the same. For example, when the first data item contains PHI, the aforementioned prefix and the suffix may appear at the beginning and at the end of the first data item such that the PHI information of the first data item is contained within the PHI presence information. Alternatively, the prefix may differ from the suffix. The PHI presence information may be comprised in a string which is either prepended or appended to the string comprising the PHI. The string may comprise alphanumeric characters and/or nonalphanumeric characters. Moreover, the same string or strings (possibly comprising alphanumeric and nonalphanumeric characters) may provide both the PHI presence information and the first PHI category information.

Similar to accessing the first PHI category information, accessing the PHI presence information may include using one or more specified regular expressions to scan the first data item and/or the first data file. The regular expressions used to access the PHI presence information may differ from the regular expressions used to access the first PHI category information. The accessing of the first PHI presence information may be implemented along with accessing the first data file and accessing the first PHI category information (e.g., as part of the same program or function call) or may be implemented separately. Accordingly, the specified regular expressions may be used to isolate or determine the PHI presence information in the first data item.

The PHI presence information may appear after the first PHI category information. For example, the PHI category information may appear at the beginning and end of data in the first data item, and the PHI presence information may appear at the beginning and end of the PHI category information.

An entry in the first data file including the first data item may also include further data items. Each of the further data items of the entry may include corresponding PHI presence information.

Use of PHI presence information may enable efficient determination of whether a given data item includes PHI. For example, a pattern corresponding to (i.e., describing) a prefix of the PHI presence information may be searched for within the first data file. Upon finding the pattern followed by a pattern corresponding to a suffix of the PHI presence information, information contained within the prefix and the suffix may be determined to be PHI.

In some cases, if the protected health information in the first data item is not to be de-identified, the method further comprises generating a third data item by modifying the first data item by deleting the PHI category information.

The modifying of the first data item may include deleting the PHI presence information in addition to the PHI category information. In this way, the third data item may display the PHI information in plain text. Accordingly, the PHI information may be used in the context of troubleshooting or testing, e.g., troubleshooting or testing a laboratory instrument.

The third data item may be stored (e.g., with other data items) in a data file for export (e.g., transmission over a computer network to a remote computing device).

Deleting the PHI category information may reduce the amount of information exported for troubleshooting, testing or analysis. This may be particularly beneficial when exporting includes sending the information over a network, since the effects on other network users may be reduced. For example, deleting the PHI category information in the context of troubleshooting may reduce the size of the file sent over a network via the removal of information that is not relevant for troubleshooting, thereby simplifying the handling and the processing of the data used for troubleshooting and expediting transmission of the data.

In some cases, the first data item comprises one or more tags. The one or more tags may comprise the first PHI category information. In addition, the one or more tags may comprise the PHI presence information.

For example, if the first data file comprises XML text, the entries of the first data file may be XML elements marked by an XML entry tag and the fields of the log file entries that comprise PHI may be XML elements marked by PHI category-dependent XML tags. Alternatively or additionally, if the first data file comprises LaTeX text, the entries of the first data file may have been marked in a LaTeX environment and the fields of the log file entries that comprise PHI may be marked with PHI category-dependent LaTeX commands.

Each tag may include metadata that enables the first data item to be efficiently found via a search. Moreover, the tags may enable efficient determination of whether the data item comprises PHI, and if so, enable the first PHI category to be efficiently determined.

Assessing whether the protected health information in the first data item is to be de-identified or not may comprise assessing whether one or more de-identification requirements are fulfilled or not. The one or more de-identification requirements may comprise a category requirement, wherein the category requirement is the requirement that the first PHI category is different from one or more given PHI categories of the plurality of PHI categories.

For example, if the one or more de-identification requirements are fulfilled, the first data item is to be de-identified. In addition or alternatively, if at least one of the de-identification requirements is not fulfilled, the first data item is not to be de-identified. When the first data item is not to be de-identified, the PHI category information may be deleted.

The use of multiple de-identification requirements, as opposed to a single de-identification requirement, may improve the flexibility and extensibility of the method for selectively de-identifying protected health information.

One of the deidentification requirements may be that a user attempting to access the first data item via a GUI does not have permission to access the first data item. In particular, this requirement in the context of the GUI.

The given PHI categories may be a proper subset of the plurality of PHI categories, i.e., the given PHI categories might not include all of the plurality of PHI categories. For example, the given PHI categories may include PHI categories related to a sample, a tube, or a laboratory instrument. Moreover, the given PHI categories may exclude PHI categories related to patient information.

The category setting may be turned on (i.e., set to true) for each of the given PHI categories. Accordingly, PHI in data items including PHI category information indicative of one of the given PHI categories may be handled differently than other PHI, as discussed above. Similarly, the category setting may be turned off (i.e., set to false) for each one of the plurality of PHI categories that is not one of the given PHI categories. Accordingly, PHI in data items including PHI category information that is not indicative of one of the given PHI categories may be handled according to a standard or default approach applicable to PHI, i.e., the PHI may be de-identified. The category setting may be configurable; for example, after the category setting is turned off PHI in the PHI category that was previously subject to different handling may be subject to default handling.

In the following example (points 1 to 8 below), the given PHI categories are limited to the sample ID category and do not include the default PHI category (the default PHI category is the category to which belong all the PHI that do not belong to the sample ID category). The example shows PHI category information and PHI presence information for default and sample ID categories in a first data file (e.g., of a laboratory instrument) and a second data file (e.g., to be exported to an external entity):
1. Patient Name (Default Category) data item in the first data file: <<<Jane Austen>>>
2. Tube barcode (sampleld Category) data item in the first data file: <<<SID_Tube_Barcode_1_5ID_>>>

Point 1 above shows a data item in the first data file including the PHI “Jane Austen”, preceded and followed by specified strings (the prefix “<<<” and the suffix “>>>”) providing the PHI presence information and the PHI category information. Hence, the data item comprises PHI that belongs to the default category, as indicated. Point 2 above shows a data item in the first data file including the PHI “Tube_Barcode_1”, which is preceded and followed by specified strings (the strings “<<<SID_” and “_SID_>>>”) providing the PHI presence information and the PHI category information. In particular, PHI category information comprised in the data item of point 2 specifies that the PHI of said data item belongs to the Sample ID category.

When the category setting for the sampleID category is set to true, the category requirement requires that the PHI category of the data item is different from the default category and, hence, the “_SID_” strings may be used to provide distinct handling of the corresponding PHI. Therefore, in a second data file generated for export (e.g., as a log file to be exported), the data items above will appear as follows:
3. Patient Name (Default Category) data item in the second data file: *****
4. Tube barcode (sampleld Category) data item in the second data file: Tube_Barcode_1

Point 3 above shows the data item from point 1 after de-identification.

Specifically, “<<<Jane Austen>>>” has been replaced with “*****”, as the data item from point 1 belongs to the default PHI category. Point 4 above shows the data item from point 2; in this case, no de-identification was performed, e.g. as the one or more de-identification requirements are not fulfilled. The data item from point 2 has been modified by deleting the PHI presence information and the first PHI category information (i.e. the strings “<<<SID_” and “_SID_>>>”).

Alternatively, the category setting for the sampleID category may be set to false. In this case, PHI category information and PHI presence information for default and sample ID categories in a first data file would be as follows:
5. Patient Name (Default Category)
data item in the first data file: <<<Jane Austen.
6. Tube barcode (Sampleld Category)
data item in the first data file: <<<SID_Tube_Barcode_1_SID_>>>

Points 5 and 6 above correspond to points 1 and 2. When the category setting for the samplelD category is set to false, then no distinct handling is applied; instead, the default approach is applied. In this case, the PHI category information is ignored and all data marked by PHI presence information, i.e., “<<<” and “>>>”, is de-identified. Therefore, when the second data file is generated (e.g., a log file is exported) the data items above will appear as follows in the second data file;
7. Patient Name (Default Category)
data item in the second data file: *****
8. Tube barcode (Sampleld Category)
data item in the second data file: *****

As shown in points 7 and 8 above, if the category setting for the samplelD category is set to false, then PHI corresponding to the sample ID category is handled the same way as other PHI, i.e., no distinct handling is applied for the sample ID category and the data item is de-identified just as the data item in the default category is de-identified.

The method may further comprise accessing de-identification data; the de-identification data may comprise information indicative of the one or more de-identification requirements.

If the one or more de-identification requirements are limited to only the category requirement, the de-identification data may specify whether PHI belonging to a PHI category (e.g., whether the PHI in the first data item belongs to the first PHI category) is to be de-identified or not.

Accessing the de-identification data may comprise retrieving a file comprising the de-identification data from the at least one memory of the computing device or from the remote data storage. Accordingly, in some cases, accessing the de-identification data includes downloading data. For example, the computing device may retrieve the file including the de-identification data in response to a user command.

Additionally or alternatively, accessing the de-identification data may comprise receiving the file comprising the de-identification data from a user or another computing device. For example, the computing device may receive the file comprising the de-identification data from another computing device via the computer network. Specifically, the file comprising the de-identification data may be received from the laboratory instrument.

Accessing the de-identification data may comprise accessing a configuration file, the configuration file comprising the de-identification data.

Storing the de-identification data in the configuration file may enable the de-identification data to be stored with other configuration data. For example, the configuration file may correspond to a logging facility of the laboratory instrument. Accordingly, the configuration file may also describe fields that could appear in an entry of the first data file in addition to the de-identification data. Combining the de-identification data with other configuration data may be more efficient than storing the de-identification data separately.

In some examples, the method may further comprise generating a second data file. In particular, if the protected health information in the first data item is to be de-identified, the second data file comprises the second data item. Additionally or alternatively, if the protected health information in the first data item is not to be de-identified, the second data file may comprise the third data item.

In addition to the second data item, the second data file may include the unprotected analysis data, e.g. data items that are comprised in the first data file and do not comprise PHI. Exemplarily, data items that do not comprise PHI are messages specifying failures of a component of a lab instrument. For instance, the string “BulkQ request exceeded maximum retry window. Failing request” may be a field of an entry of the first data file that (i) does not comprise PHI and (ii) specifies that a software component, BulkQ, has failed because of too many retries of sending data to the cloud.

The second data file may be the data file for export. Accordingly, the second data file may be sent over the computer network to the remote computing device. In this way, the unprotected analysis data in the second data file may be analyzed without providing access to the PHI.

The method may further comprise accessing file information indicative of a file category. Assessing whether the PHI of the first data item is to be de-identified or not may be based on the file information. In particular, the file category is the category to which the second data item belongs or the category to which the file (e.g., the second data file) including the second data item belongs. Alternatively, the file category is the category to which the third data item belongs.

The one or more de-identification requirements may comprise a file category requirement. The file category requirement may correspond to a destination of the second data file (e.g., the destination of the second data item). For example, a first file category may correspond to a closed network for analysis and troubleshooting that is relatively secure and a second file category may correspond to a public network that is relatively insecure. Accordingly, upon export of the second data file, the second data file may be sent to the destination via the computer network.

Hence, the file category requirement may make it easy to reveal selected PHI for analysis (e.g., troubleshooting or testing) in a relatively secure destination while ensuring that the same PHI is de-identified when the second data file is to be sent to an insecure destination.

Similar to the destination, the file category may correspond to a target user of the second data item. For instance, if the target user is a field engineer or someone else concerned with troubleshooting, Sample ID information may be needed (i.e., the samplelD might not be de-identified), whereas if the target user is a typical user who is not concerned with troubleshooting, the PHI does not need to be readable and can be de-identified.

Accordingly, the file category information may make it easy to reveal selected PHI to target users at a destination when the target users have a need-to-know the selected PHI. In the absence of a need-to-know for the target user at the destination, the same PHI may be de-identified.

Alternatively or additionally, the file category requirement may correspond to a type of file. For example, a third file category may correspond to a file for troubleshooting the workflow of the biological samples processed by an automated laboratory system.

Exemplarily, in the files belonging to the third file category, PHI belonging to the sample ID shall not be de-identified, e.g., so that the route of the samples in the automated laboratory system can be reconstructed.

In contrast to the third file category, a fourth file category, instead, may correspond to a file for assessing the down time or the idle time of the laboratory instruments of an automated laboratory system. In the files belonging to the fourth category, PHI belonging to the sample ID is not relevant in the assessment of the instruments idle time and may be de-identified. Hence, the file category improves flexibility in the de-identification of PHI, enabling the same PHI to be treated differently in different files, if the file category so indicates.

According to a second aspect, a computer-implemented method for generating a first data item is provided. The method comprises obtaining protected health information, the protected health information belonging to a first PHI category of a plurality of PHI categories. The method further comprises determining the first PHI category. The method further comprises generating, based on the first PHI category, the first data item, the first data item comprising the protected health information and category information, the category information being indicative of the first PHI category, wherein the first data item is part of a first data file. In particular, the PHI comprised in the first data item is not in de-identified form.

Similar to the first aspect, the method may be carried out by one or more computing devices. A computing device may comprise at least one memory and at least one processor. A computing device may also comprise one or more input/output units. The computing device may be or be part of a laboratory instrument.

Obtaining the protected health information may comprise retrieving the protected health information from at least one memory of the computing device. In addition, or alternatively, obtaining the protected health information may include receiving the protected health information from a user. For example, the laboratory instrument may retrieve the protected health information from memory (e.g., for protected health information corresponding to the laboratory instrument itself, such as a tube ID or a barcode) or receive the protected health information via user input (e.g., for patient or doctor related information such as patient gender or date of birth). Obtaining the protected health information may comprise receiving the protected health information e.g. from a laboratory instrument.

The first PHI category may be determined based on the risk of whether the protected health information can be used to arrive at the identity of an individual associated with the protected health information. In addition, the first PHI category may be determined based on whether the protected health information may be useful for troubleshooting. Exemplarily, determining the first PHI category may be carried out by executing computer instructions that specify the PHI category, to which the protected health information belongs. In this example, the determining the first PHI category may include providing the first PHI category.

For example, if the risk that the protected health information can be used to arrive at the identity of the individual associated with the protected health information is relatively low (i.e., low reidentification risk) and the protected health information appears to be useful for troubleshooting then the first PHI category may indicate that the protected health information is to be handled differently than other protected health information. Conversely, if the reidentification risk is relatively high and the protected health information does not appear to be useful for troubleshooting then the first PHI category may indicate that the protected health information is to be handled according to a default approach for handling protected health information.

Accordingly, the default approach may include de-identifying the protected health information, e.g., irreversibly. Furthermore, the different handling may include performing no de-identification of the protected health information or performing reversible de-identification of the protected health information (e.g., via a code or encryption as discussed above).

Exemplarily, within a PHI-handling framework comprising the methods of some embodiments of the disclosed technology, any field, property or function parameter identified as sensitive and needing protection is marked as PHI, e.g. by using a PHI attribute, in particular a C #attribute. The PHI-handling framework may log (e.g., at runtime) and display information marked as PHI in the first data file such that sensitive data may be, if needed, concealed. For example, a marked PHI attribute may be written to the first data file as “<<< {phi_value}>>>”, where the PHI is shown as “{phi_value}” and the PHI presence information is shown as the three “<” symbols preceding the PHI and the three “>” symbols following the PHI. For example, any PHI attribute marked as PHI may, if needed, be anonymized and displayed as a series of asterisks.

In particular, the PHI-handling framework may be configured to carry out the method according to the second aspect of the disclosed technology. More particularly, the PHI-handling framework is a computer program product comprising instructions which, when the PHI-handling framework is executed by a computer, cause the computer to carry out the steps of the method according to the second aspect of the disclosed technology. The PHI-handling framework may include one or more classes and may be integrated into a more general logging framework.

The PHI-handling framework may obtain PHI from a laboratory instrument by receiving information specifying an event that occurred in the laboratory instrument. For instance, the laboratory instrument may send to the PHI-handling framework information specifying that a tube order for a given tube (say the tube with identification number “Tube001”) is queried for a given patient (say Ms. Muholland).

This information on the event may be processed by the PHI-handling framework by using the following code:

Log.lnfo(“Tube order for tube id {OJ is queried for patient
{1}”, PHILogValue.Log(tubeID, new
DefaultPHIValueFormatter(PH/Category.SampleID)),
PHILogValue.Log(patientName))

Hence, the PHI-handling framework uses the information received from the laboratory instrument to determine the template of the text of the log entry, i.e. the string “Tube order for tube id {OJ is queried for patient {1J” and the information that, in the first data file, shall replace the placeholders {0} and {1}. Specifically, a data item comprising information identifying the sample tube shall replace the placeholder {0} and a data item comprising patient name information shall replace the placeholder {1}. Accordingly, as in the code above, {0} is processed according to a first code snippet:

PHILogVa/ue.Log(order.SamplelD, new
DefaultPHIValueFormatter(PHICategory,Sample ID)).

In the first code snippet, the field identifier, “tubelD” from the code above has been replaced by the variable “order.SamplelD”, which has a value corresponding to “tubeID”. The PHI category to which the PHI comprised in the string “Tube001” belongs is determined by executing the first code snippet (i.e., the PH/Log Value.Log function). In particular, the first code snippet specifies that the PHI comprised in the string “Tube001” belongs to the sample ID category (cf. the argument of the DefaultPHIValueFormatter method). In the first code snippet, the PHI category of samplelD is simply provided. However, the PHI category may also be determined according to more complex instructions, e.g., based on the value of the samplelD in a data item. For example, if the samplelD corresponds to a sample ID belonging to a set of test samples, the samplelD may be assigned to a test category, whereas other samplelDs not belonging to the set of test samples may be assigned to the samplelDcategory.

As in the code above, {1} is processed according to a second code snippet:
PHILogVa/ue.Log (order.Patient,PatientDisplayName).

In the second code snippet, the PHI category patientName has been replaced by the variable order, PatientPatientDisplayName, which corresponds to the PHI category. The PHI category to which the PHI comprised in the string “Muholland “belongs is determined by executing the second code snippet The second code snippet specifies that the string “Muholland “comprises information related to the patient identity (cf. order. Patient. PatientDisplayName) and e.g. belongs to the default category.

The PHILogValue.Log method is configured to generate, based on the PHI category of the detected PHI, data items comprising the protected health information and PHI category information. In particular, the following entry is generated for a data file (e.g., the first data file):

Tube order for tube id<<<SID_Tube001_SID_>>> is queried for patient <<<Mulholland>>>

Accordingly, the data item including the sample ID “Tube001” now further includes PHI category information corresponding to the sample ID PHI category, while the data item including the patient “Mulholland “includes PHI category information corresponding to the default PHI category.

Directly after generating the first data item based on the first PHI category, the protected health information in the first data item might not be in de-identified form. More specifically, de-identification of the first data item may occur upon export of the first data item, e.g., export of the first data file including the first data item.

Generating the first data item based on the first PHI category ensures the efficiency of selective de-identification of the protected health information of the first data item. Specifically, since the first data item comprises the category information, regular expressions can be efficiently used to access the category information; the category information can then be used to determine if the PHI in the first data item is to be de-identified.

The method may further comprise assessing whether the protected health information meets at least one criteria for protected health information. A first of the criteria for protected health information may specify that the protected health information includes at least one direct identifier.

A second of the criteria for protected health information may specify that the protected health information includes at least one indirect identifier. Alternatively, the second of the criteria for protected health information may specify that the protected health information includes at least two direct identifiers and a third of the criteria for protected health information may specify that the protected health information includes at least one indirect identifier from a list of high risk indirect identifiers.

According to some embodiments, the first data file may be a log file of a laboratory instrument, e.g. an automated laboratory system. Accordingly, the first data item may be generated by the laboratory instrument as part of an entry for the log file. For example, the protected health information may be a sample ID and the laboratory instrument may obtain the sample ID from memory and insert the sample ID at the end of a log file entry indicating that an aliquot tube has been created.

Entries in the log file may be created on the laboratory instrument but selective de-identification might not happen immediately. Instead, PHI in log file data items may be selectively de-identified before export, e.g., via a network to a remote computer (e.g., a third party or foreign entity). The PHI in the data items may be marked, e.g., with tags as described above, so that the PHI can be identified (e.g., via regular expressions) prior to export of the data items.

Generally, an automated laboratory system (also referred to as: “ALS”) is an assembly comprising a plurality of components and a computing device, wherein the computing device is operatively connected to these components and is configured to control each component. A component may be an analytic instrument, a pre-analytic instrument, a post-analytic instrument, an input/output module, a transportation component (e.g. track, belt, tube carrier) configured to move a sample.

An analytic instrument (e.g. analyzer) is an instrument configured to carry out one or more analytic steps, such as measuring one or more characteristics of the sample, e.g. the concentration of a given analyte. A pre-analytic instrument is an instrument configured to carry out one or more pre-analytic steps on a biological sample to prepare said sample for the analytic instrument(s). For example, centrifuges, aliquoters and de-cappers are pre-analytic instruments. A post-analytic instrument is an instrument configured to carry out one or more post-analytic steps on the biological sample after the sample has been processed by one or more analytic instruments. For example, re-cappers and sample storage units are post-analytic instruments. The terms “instrument” and “laboratory instrument” will be used to generally refer to an analytic instrument, a pre-analytic instrument or a post-analytic instrument.

Hence, the laboratory instrument can create a log file in which protected health information is categorized such that selective de-identification of the protected health information can be performed in an efficient manner.

According to some embodiments, the first PHI category may specify that the protected health information refers to a biological sample, to patient data, to doctor data or to laboratory test data.

More specifically, the first PHI category may specify that the protected health information refers to one of the following categories: SamplelD, PatientID, DoctorlD, Test. For example, PHI uniquely identifying a sample, PHI uniquely identifying the tube within which the sample is collected, and/or PHI uniquely identifying the secondary sample obtained by aliquoting a sample belong to the SamplelD category. For instance, PHI specifying the first name of a patient, PHI specifying the middle name of a patient, PHI specifying the surname of a patient, PHI specifying the salutation of a patient, PHI specifying the gender of a patient, PHI specifying the birth date of a patient, and/or PHI specifying the address of a patient belong to the PatientID category. Exemplarily, PHI specifying the identity of the doctor that has ordered the tests and/or PHI specifying the identity of the doctor that attends to a patient belong to the Doctorl D category. As another example, a set of identifiers (e.g., samplelDs, TubelDs, SpecimenlDs) designated for testing of laboratory instruments may belong to the Test category.

According to a third aspect, a computing device is provided. The computing device comprises a processor configured to perform the method according to the first aspect of the disclosed technology and/or the method according to the second aspect of the disclosed technology. According to a fourth aspect, a laboratory instrument is provided. The laboratory instrument comprises the computing device provided above.

According to a fifth aspect, a computer program product is provided. The computer program product comprises instructions which, when the program is executed by a computer, cause the computer to carry out the steps of the method according to the first aspect of the disclosed technology and/or the steps of the method according to the second aspect of the disclosed technology.

According to a sixth aspect, a computer-readable storage medium is provided. The computer readable storage medium comprises instructions which, when executed by a computer, cause the computer to carry out the steps of the method according to the first aspect of the disclosed technology and/or the steps of the method according to the second aspect of the disclosed technology. The computer readable storage medium may be non-transitory, i.e. tangible.

The subject matter described in this disclosure can be implemented as a method or on a device, possibly in the form of one or more computer programs (e.g., computer program products). Such computer programs may cause a data processing apparatus to perform one or more operations described in the present disclosure.

The subject matter described in the present disclosure can be implemented in a data signal or on a machine readable medium, where the medium is embodied in one or more information carriers, such as a CD-ROM, a DVD-ROM, a semiconductor memory, or a hard disk. In particular, disclosed subject matter may be tangibly embodied in a machine (computer) readable medium.

In addition, the subject matter described in the present disclosure can be implemented as a system including a processor, and a memory coupled to the processor. The memory may encode one or more programs to cause the processor to perform one or more of the methods described in the application. Further subject matter described in the present disclosure can be implemented using various machines.

Details of one or more implementations are set forth in the exemplary drawings and description that follow. Other features will be apparent from the description, the drawings, and from the claims.

### BRIEF DESCRIPTION OF THE DRAWINGS

FIG. 1 shows a schematic representation of a portion of a first data item.

FIG. 2 shows a further schematic representation of a portion of a first data item.

FIG. 3 shows a first method for selectively de-identifying protected health information.

FIG. 4A shows steps of a second method for selectively de-identifying protected health information.

FIG. 4B continues the second method for selectively de-identifying protected health information.

FIG. 5 shows a method for generating a first data item.

### DETAILED DESCRIPTION

In the following text, a detailed description of examples will be given with reference to the drawings. Various modifications to the examples may be made. In particular, one or more elements of one example may be combined and used in other examples to form new examples.

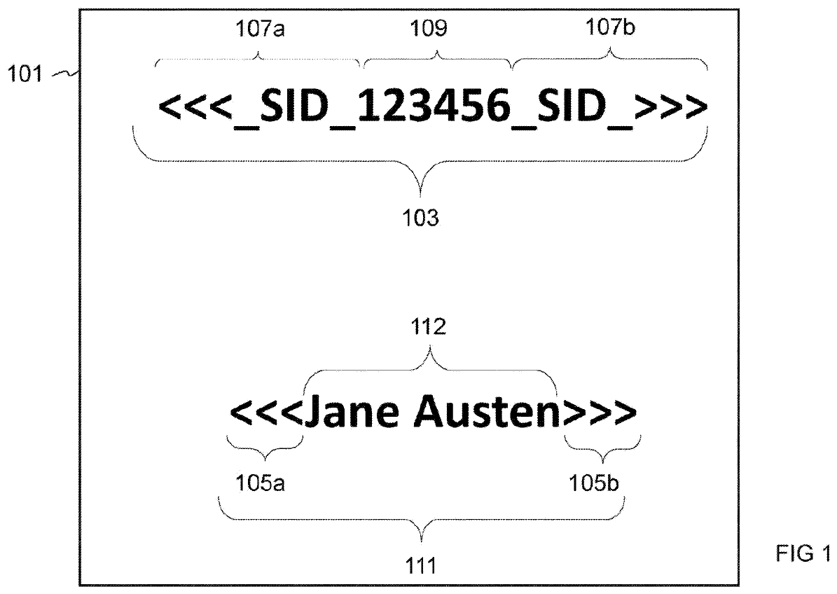

FIG. 1 shows a schematic representation of a portion of a first data file 101 including analysis data; in particular, the first data file 101 includes a first data item 103. The first data item 103 may be a string of characters. The first data item 103 comprises a numeric string 109 with the protected health information (PHI). In this example, the PHI specifies the ID of a sample processed by a laboratory instrument (not shown).

The first data item 103 includes protected health information (PHI) presence information. The PHI presence information may be indicative of whether the first data item 103 comprises protected health information, as shown in a string 109. Hence, the string 109 includes the protected health information “123456”. The PHI presence information may include one or more symbols, e.g., at least two symbols. The symbols may be nonalphanumeric characters.

In the example of FIG. 1, string “<<<SID_” 107a at the beginning of the first data item 103 and string “_SID_>>>” 107b at the end of the first data item 103 provide both the PHI presence information and the first PHI category information. In this example, the presence of PHI and the category to which the PHI belongs are specified by a prefix—the string “<<<SID_” 107a—and a suffix (i.e., postfix)—the string “_SID_>>>” 107b of the first data item 103. In addition, the prefix and the suffix differ, since three “<” characters appear in the prefix and three “>” characters appear in the suffix.

In some cases, the suffix 107b and the prefix 107a may include one or more alphanumeric characters and one or more nonalphanumeric characters (e.g., at least two alphanumeric characters and at least two nonalphanumeric characters). In the example of FIG. 1, the both the suffix 107b and the prefix 107a include three alphanumeric characters (that constitute the string “SID”) and two underscores. The suffix 107b and the prefix 107a further include three characters “>” and three “<” characters, respectively.

In the example of FIG. 1, the PHI presence information specifies that the first data item 103 comprises PHI; the first PHI category information, comprised in the prefix 107a and the suffix 107b, indicates that the protected health information belongs to the sample ID category. The sample ID category may be one of one or more given PHI categories. For example, the given PHI categories may be limited to the sample ID category. Protected health information in the sample ID category may be handled differently than other protected health information. All protected health information that does not fall in the sample ID category may be in a default category. Specifically, protected health information that does not correspond to one of the other PHI categories may fall in the default category. Protected health information in the default category may be de-identified, e.g., via an irreversible replacement. Accordingly, such protected health information may be displayed as a series of symbols (e.g., asterisks, such as “*****”) in a graphical user interface or marked with symbols (e.g., the characters “>” and “<”) in log files.

In the example of FIG. 1, the first data file 101 includes a further data item 111. The data item 111 comprises protected health information which is specified by an alphanumeric string 112 including the name “Jane Austen”; the alphanumeric string 112 represents the name of a patient. The data item 111 includes a prefix, i.e. the string “<<<” 105a, and a suffix, i.e. the string “>>>” 105b, which provide the PHI presence information and, in particular, specify that the data item 111 comprises PHI. The prefix and the suffix further specify that the PHI in the data item 111 belongs to the default PHI category, i.e. to the category to which all the PHI that do not belong to the sample ID category belong.

Alternatively, the given PHI categories may include multiple PHI categories. For example, an additional patient name category may be one of the given PHI categories. In particular, PHI identifying the first name of the patient, PHI identifying the surname of the patient, and/or PHI identifying the middle of the patient belong to the patient name category

In this example, each of the patient name and sample ID categories may enable distinct handling for protected health information corresponding to those categories.

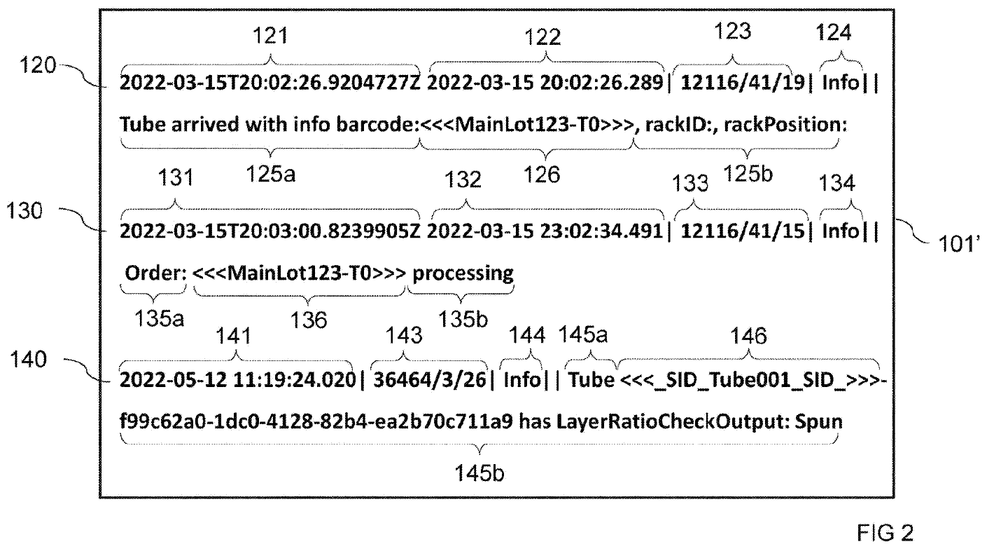

FIG. 2 shows a further schematic representation of a portion of a first data file 101′. The portion of the first data file 101′ shown in FIG. 2 comprises a first entry 120, a second entry 130 and a third entry 140, The first entry 120 and the second entry 130 each include the following fields: an end timestamp 121, 131 (e.g., 2022-03-15T20:02:26.9204727Z), a start timestamp 122, 132 (e.g., 2022 Mar. 15 20:02:26.289), an identifier 123, 133 (e.g., 12116/41/19), a severity level 124, 134 (e.g., Info), descriptive text 125a, 125b, 135a, 135b (e.g., Tube arrived with info barcode), and data items including PHI category information and PHI presence information 126, 136 (e.g., <<<Mainlot123-T0>>>). According to the PHI category information and the PHI presence information provided by the data items 126 and 136 (i.e. by the strings “>>>” and “<<<” thereto), the data items 126, 136 comprise PHI belonging to the default category.

The third entry 140 includes a timestamp 141, an identifier 143, a severity level 144, descriptive text 145a, 145b, and a data item including PHI category information and PHI presence information 146 (i.e. the string “<<<SID_Tube001_SID_>>>”).

In particular, the alphanumeric string “f99c62a0-1 dc0-4128-82b4-ca2b70c711a9” comprised in the string 145a is an identifier that uniquely identifies an operation of the laboratory instrument. According to the PHI category information and the PHI presence information provided by the data item 146 (i.e. by the strings “_SID_>>>” and “<<<SID_” thereto), the data item 146 comprises PHI belonging to the sample ID category.

The first data file 101′ may be stored on a laboratory instrument or may be in storage controlled by the laboratory instrument. Hence, PHI in the data items 126 and 136 is still readable and has not been replaced or obscured. Before the first data file 101′ is exported (e.g., to a remote computer via a computer network), the data items 126 and 136 may be de-identified, e.g., replaced with symbols such as asterisks.

The entries of the first data file 101′ may have more or fewer fields than the entries 120, 130, 140 shown in FIG. 2. Data of the entries 120, 130, 140 that is not marked with PHI presence information may be unprotected analysis data.

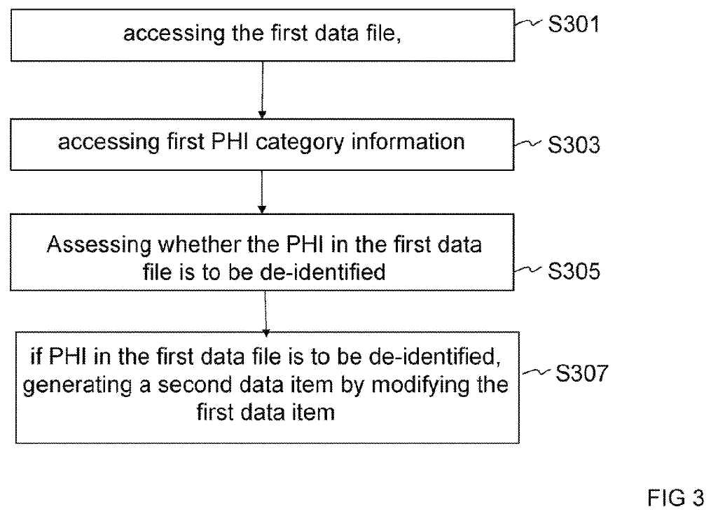

FIG. 3 shows a first method for selectively de-identifying protected health information. At step S301, the first data file 101 is accessed. The first data file 101 comprises at least the first data item 103. The first data item 103 comprises the PHI and the first PHI category information. The first PHI category information is indicative of a first PHI category of a plurality of PHI categories. The PHI in the first data item 103 belongs to the first PHI category.

Accessing the first data file 101 may comprise retrieving the first data file 101 from the at least one memory of the computing device or from remote data storage (e.g., a database, secondary memory, cloud storage or the like). Accordingly, in some cases, accessing the first data file 101 may comprise downloading data. For example, the computing device may retrieve the data file in response to a user command.

Additionally or alternatively, accessing the first data file 101 may comprise receiving the first data file 101, e.g., from a user or another computing device. For example, the computing device may receive the first data file 101 from another computing device via a computer network. Specifically, the first data file 101 may be received from a laboratory instrument. The laboratory instrument may be an analyser, i.e., an apparatus configured to carry out one or more clinical tests on a biological sample. More specifically, the laboratory instrument may be a chemistry or hematology analyzer. The two options for accessing the first data file 101 are not mutually exclusive. For example, accessing the first data file 101 may include receiving the first data file 101, during the first data file 101 in the memory of the computing device and retrieving the first data file 101 by accessing the memory.

At step S303, the first PHI category information is accessed. Accessing the first PHI category information may include using one or more specified regular expressions to scan the first data item 103 and/or the first data file 101. The accessing of the first PHI category information may be implemented along with accessing the first data file 101 (e.g., as part of the same program or function call) or may be implemented separately. A regular expression may include a sequence of characters (e.g., metacharacters and regular characters) specifying a search pattern to describe and/or parse text. Accordingly, the specified regular expressions may be used to isolate or determine the first PHI category information in the first data item 103.

At step S305, the method comprises assessing, based on the first PHI category information, whether the PHI in the first data item 103 is to be de-identified or not; the assessing may include using the first PHI category information to determine whether the PHI in the first data item 103 is to be de-identified. Accordingly, a category setting may be associated with the first PHI category information. If the category setting is turned off (i.e., set to false), then the first data item 103 might be de-identified. Similarly, if no category setting is associated with the first PHI category information, then the first data item 103 might be de-identified. Alternatively, if the category setting is turned on (i.e., set to true), then the first data item 103 might be handled differently (i.e., distinctly) from other PHI. For example, when the category setting is turned on, the first data item 103 might not be de-identified. Alternatively, when the category setting is turned on, the first data item 103 might be de-identified in such a way that it is possible to reconstruct the original data after de-identification. For example, when the category setting is turned on, the first data item 103 might be encrypted.

At step S307, if the PHI in the first data item 103 is to be de-identified, the method further comprises generating a second data item (not shown) by modifying the first data item 103 such that the protected health information is de-identified. Hence, step S307 may include de-identifying the first data item 103 by generating the second data item, wherein the second data item corresponds to the first data item 103 except that the PHI of the first data item 103 has been replaced or obfuscated in the second data item.

For example, the PHI of the first data item 103 may be replaced with a specified (e.g., predefined) string in the second data item, such that the specified string cannot be used to reconstruct the original PHI. Accordingly, PHI having the PatientName category in the first data item 103 may be replaced with the specified string including four asterisks, i.e. the string “*****” in the second data item. In the example, since all data items in the category are replaced with the same specified string, the second data item cannot be used as a basis for arriving at the first data item 103; hence, the replacement is irreversible.

Alternatively, generating the second data item may include modifying the first data item 103 by encrypting the PHI of the first data item 103. As yet another alternative, the first data item 103 may be replaced with a code that can be decoded by a third party (e.g., a doctor) possessing a key. For example, a patient name may be replaced with a corresponding coded name that can be decoded by the third party using a table matching patient names to corresponding coded names. In the preceding two examples, the replacement is reversible, i.e., the second data item can be used as a basis for arriving at the first data item 103.

De-identification may be handled differently depending on a destination of the second data item. For example, the second data item may be exported to the GUI or to a second data file. When exported to the GUI, the PHI may be de-identified (e.g., obfuscated) by using a series of symbols, e.g., asterisks. When exported to the second data file, the PHI may be de-identified by replacing the PHI with a specified series of characters.

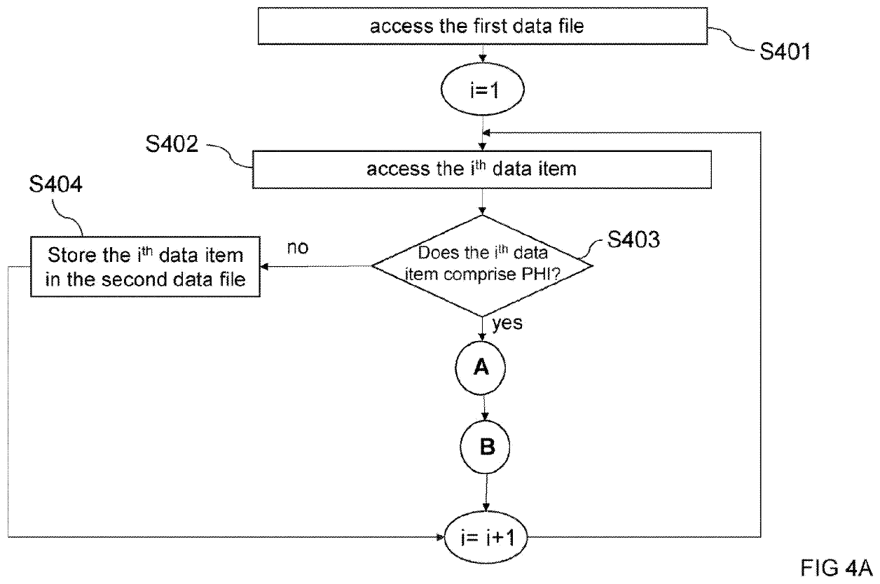

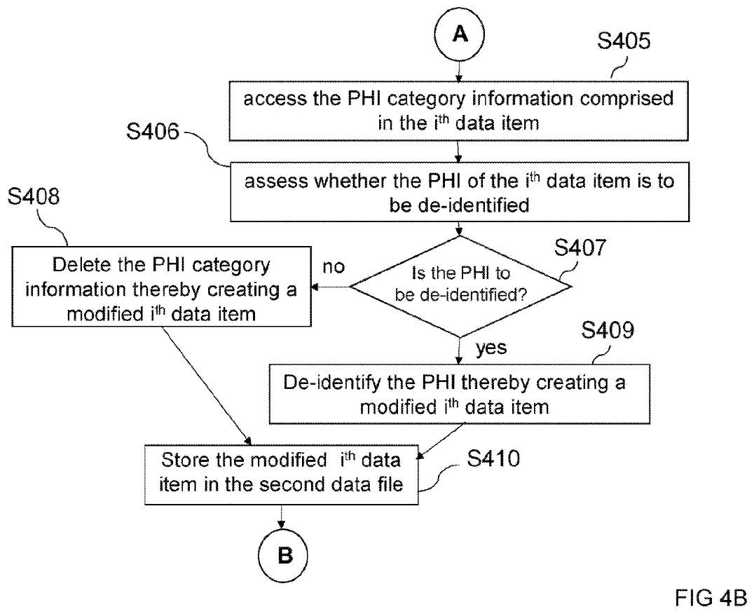

FIG. 4A shows steps of a second method for selectively de-identifying protected health information.

At step S401, the second method comprises accessing the first data file 101, 101′. Accessing the first data file 101, 101′ may comprise retrieving the first data file 101, 101′ from the at least one memory of the computing device or from remote data storage (e.g., a database, secondary memory, cloud storage or the like). Accordingly, in some cases, accessing the first data file 101, 101′ may comprise downloading data. For example, the computing device may retrieve the data file in response to a user command.

Additionally or alternatively, accessing the first data file 101, 101′ may comprise receiving the first data file 101, 101′, e.g., from a user or another computing device. For example, the computing device may receive the first data file 101, 101′ from another computing device via a computer network. Specifically, the first data file 101, 101′ may be received from a laboratory instrument.

The first data file 101, 101′ includes a plurality of data items. The first data file 101, 101′ may include a plurality of entries. Each entry may include one or more data fields or components. Each field may include one or more data items. Each entry in the first data file 101, 101′ may have a corresponding structure (e.g., the same number of fields, fields having the same data type). Unless otherwise indicated, information provided below with regard to the first data item 103 may also be applicable to the other data items in the first data file 101, 101′. After step S401, a counter i may be set to 1.

At step S402, the second method comprises accessing the i^{th} data item. Since the counter i is set to 1, the first time step S402 is carried out, accessing the i^{th} data item corresponds to accessing the first data item 103.

At step S403, the second method comprises determining whether the i^{th} data item comprises PHI. Determining whether the jfh data item comprises PHI may include accessing PHI presence information in the first data file 101, 101′ (e.g., PHI presence information of the first data item 103), the PHI presence information being indicative of whether the i^{th} data item comprises protected health information and assessing, based on the PHI presence information, whether the i^{th} data item comprises protected health information or not.

When the i^{th} data item does not comprise PHI, step S404 is carried out.

Accordingly, the i^{th} data item is stored in the second data file. In this case, the i^{th} data item may include or consist of unprotected analysis data. After step S404, the counter i is incremented and step S402 is carried out for the (i+1)^{th} data item.

When the j^{fh} data item comprises PHI, steps of FIG. 4B are carried out, starting with step S405, as discussed in more detail below.

FIG. 48 continues the second method for selectively de-identifying protected health information.

At step S405, the second method may further comprise accessing PHI category information comprised in the lh data item (hereinafter also referred to: “i^{th} PHI category information”). The i^{th} PHI category information comprised in the i^{th} data item indicates the PHI category, to which the PHI comprised in the i^{th} data item belong, i.e., the PHI category of the i^{th} data item. For example, the i^{th} PHI category information may distinguish data items' PHI belonging to the PHI category of the i^{th} data item from PHI of other data items that does not belong to the PHI category of the i^{th} data item. The i^{th} PHI category information, may be comprised in a string of characters detectable by a parser, e.g., via one or more regular expressions (i.e., pattern matching). The PHI category information of the i^{th} data item may be provided by a prefix and/or a suffix of the data item, as described above and shown in FIG. 1 and FIG. 2 above. Alternatively, the PHI category information of the i^{th} data item may be comprised in a string which is either prepended or appended to the string comprising the PHI.

At step S406, the second method may further comprise assessing, based on the PHI category information of the i^{th} data item, whether the PHI of the i^{th} data item is to be deidentified. In particular, the PHI category information of the i^{th} data item is used to determine whether the PHI in the i^{th} data item is to be de-identified. Accordingly, a category setting may be associated with the PHI category information of the i^{th} data item. If the category setting is turned off (i.e., set to false), then the i^{th} data item might be de-identified. Similarly, if no category setting is associated with the PHI category information, then the i^{th} data item might be de-identified.

Alternatively, if the category setting is turned on (i.e., set to true), then the i^{th} data item might be handled differently (i.e., distinctly) from other PHI. For example, when the category setting is turned on, the i^{th} data item might not be de-identified.

Alternatively, when the category setting is turned on, the i^{th} data item might be de-identified in such a way that it is possible to reconstruct the original data after de-identification. For example, when the category setting is turned on, the i^{th} data item might be encrypted. For instance, step S406 may be carried out by assessing whether one or more de-identification requirements are fulfilled or not. The one or more de-identification requirements comprises the category requirement, i.e. the requirement that the first PHI category is different from one or more given PHI categories of the plurality of PHI categories.

Exemplarily, the given PHI categories are limited to the sample ID category and do not include the default PHI category, i.e. the category to which belong all the PHI that do not belong to the sample ID category. Accordingly, the PHI comprised in the i^{th} data item is de-identified if it belongs to the default PHI category and the PHI comprised in the i^{th} data item is not de-identified if it belongs to the sample ID. For instance, if the first data item is the data item 101′ depicted in FIG. 2, the data items 126 and 136 are to be de-identified, while the data item 146 shall not be de-identified.

At step S407, based on the assessment of step S406, a determination is made regarding further steps carry out for the second method.

If the PHI is not to be de-identified, e.g. if the PHI belongs to the sample ID category, the second method may further comprise deleting the Jlh PHI category information and thereby creating a modified jfh data item at step S408. For example, if the first data item is the data item 101′ depicted in FIG. 2, at step S407 the data item 146 is modified by deleting the prefix “<<<SID_” and the suffix “_SID_>>>”, so that the modified data item is the string “Tube001”. The creating of the modified jfh data item may include deleting the PHI presence information in addition to the PHI category information. In this way, the modified i^{th} data item may display the PHI in plain text. Accordingly, the PHI may be used in the context of troubleshooting or testing, e.g., troubleshooting or testing a laboratory instrument.

The modified i^{th} data item may be in the second data file for export (e.g., transmission over a computer network to a remote computing device).

If step S407 leads to a determination that the PHI is to be de-identified, then the PHI may be deidentified to create a modified i^{th} data item at step S409. De-identifying the PHI may include modifying or obfuscating the PHI.

For example, the PHI and the i^{th} PHI category information may be replaced with a specified (e.g., predefined) string, i.e. the string “****”, such that the specified string cannot be used to reconstruct the original PHI. For example, if the first data item is the data item 101′ depicted in FIG. 2, at step S407 the data items 126 and 136 are de-identified by substituting them with the string “****”. In particular, if the i^{th} data item comprises PHI related to the patient identity, i.e. belonging to the default PHI category, the PHI of the i^{th} data item is replaced with the specified string including four asterisks, i.e. the string “****”, in the i^{th} data item, so that the modified i^{th} data item is the string “****”. In the example, since all data items in the default PHI category are replaced with the same specified string, the i^{th} data item cannot be used as a basis for arriving at the first data item 103; hence, the replacement is irreversible. The specified string may include alphanumeric and/or nonalphanumeric characters. Alternatively, the specified string may be limited to nonalphanumeric characters.

Alternatively, de-identifying the i^{th} data item may include encrypting the PHI of the i^{th} data item. As yet another alternative, the PHI of the i^{th} data item may be replaced with a code that can be decoded by a third party (e.g., a doctor) possessing a key. For example, a patient name may be replaced with a corresponding coded name that can be decoded by the third party using a table matching patient names to corresponding coded names. In the preceding two examples, the replacement is reversible, i.e., the modified i^{th} data item can be used as a basis for arriving at the i^{th} data item.

At step S410, the second method comprises storing the modified i^{th} data item in the second data file. Step S410 may follow step S408 or step S409. Accordingly, the second data file may include data items with de-identified PHI and data items with PHI that has not been deidentified.

After step S410, the second method returns to FIG. 4A and the counter i is incremented before returning to step S402.

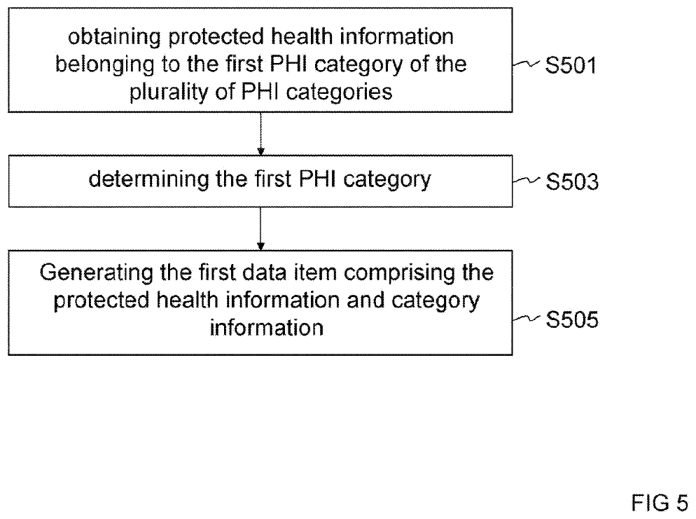

FIG. 5 shows a method for generating a first data item. At step S501, the method comprises obtaining protected health information, the protected health information belonging to a first PHI category of a plurality of PHI categories. Obtaining the protected health information may comprise retrieving the protected health information from at least one memory of the computing device. At step S503, the first PHI category is determined. At step S505, the method further comprises generating, based on the first PHI category, the first data item, the first data item comprising the protected health information and the category information. The category information is indicative of the first PHI category, wherein the first data item is part of the first data file.

Directly after generating the first data item based on the first PHI category, the protected health information in the first data item is not in de-identified form. More specifically, de-identification of the first data item occurs upon export of the first data item, e.g., export of the first data file including the first data item.

In particular, the method for generating the first data item shown in FIG. 5 may be carried out by the PHI-handling framework described above. The PHI-handling framework may obtain PHI from a laboratory instrument by receiving information specifying an event that occurred in the laboratory instrument (step S501). For instance, the laboratory instrument may send to the PHI-handling framework information specifying that a tube order for a given tube (say the tube with identification number “Tube001”) is queried for a given patient (say Ms. Muholland).

This information on the event may be processed by the PHI-handling framework by using the following code:

Log.lnfo(“Tube order for tube id {OJ is queried for patient {1}”,
PHILogValue.Log(tubeID, new
DefaultPHIValueFormatter(PHICategorySampfe/O)),
PHILogValue.Log(patientName))

Hence, the PHI-handling framework uses the information received from the laboratory instrument to determine the template of the text of the log entry, i.e. the string “Tube order for tube id {OJ is queried for patient {1}” and the information that, in the first data file, shall replace the placeholders {0} and {1}. Specifically, a data item comprising information identifying the sample tube shall replace the placeholder {0} and a data item comprising patient name information shall replace the placeholder {1}. Accordingly, as in the code above, {0} is processed according to a first code snippet:

PHILogVa/ue.Log(order.SamplelD,new
DefaultPHIValueFormatter(PHICategory SampleID)).

In the first code snippet, the field identifier, “tubelD” from the code above has been replaced by the variable “order.SamplelD”, which has a value corresponding to “tubeID”. The PHI category to which the PHI comprised in the string “Tube001” belongs is determined by executing the first code snippet (i.e., the PH/Log Value.Log function). In particular, the first code snippet specifies that the PHI comprised in the string “Tube001” belongs to the sample ID category (cf. the argument of the DefaultPHIValueFormatter method). In the first code snippet, the PHI category of samplelD is simply provided. However, the PHI category may also be determined according to more complex instructions, e.g., based on the value of the samplelD in a data item. For example, if the samplelD corresponds to a sample ID belonging to a set of test samples, the samplelD may be assigned to a test category, whereas other samplelDs not belonging to the set of test samples may be assigned to the samplelD category.

As in the code above, {1} is processed according to a second code snippet:
PHILogValue.Log (order.Patient.PatientDisplayName).
   In the second code snippet, the PHI category patientName has been replaced by the variable order. Patient. PatientDisplayName, which corresponds to the PHI category. The PHI category to which the PHI comprised in the string “Muholland “belongs is determined by executing the second code snippet. The second code snippet specifies that the string “Muholland “comprises information related to the patient identity (d. order. Patient. PatientDisplayName) and e.g. belongs to the default category.

The PHILogValue.Log method is configured to generate, based on the PHI category of the detected PHI, data items comprising the protected health information and PHI category information. In particular, the following entry is generated for a data file (e.g., the first data file):
Tube order for tube id <<<SID_Tube001_SID_>>> is queried for patient <<<Mulholland>>>

Accordingly, the data item including the sample ID “Tube001” now further includes PHI category information corresponding to the sample ID PHI category, while the data item including the patient “Mulholland “includes PHI category information corresponding to the default PHI category.

1. Computer implemented method for selectively de-identifying protected health information, PHI, the method comprising:
accessing a first data file, the first data file comprising at least a first data item, wherein the first data item comprises PHI and first PHI category information, wherein the first PHI category information is indicative of a first PHI category of a plurality of PHI categories, the PHI in the first data item belonging to the first PHI category;
accessing the first PHI category information;
assessing, based on the first PHI category information, whether the PHI in the first data item is to be de-identified or not; and
if the PHI in the first data item is to be de-identified,
generating a second data item by modifying the first data item such that the protected health information is de-identified.

2. Method according to claim 1, further comprising:
accessing PHI-presence information in the first data file, the PHI-presence information being indicative of whether the first data item comprises protected health information; and
assessing, based on the PHI-presence information, whether the first data item comprises protected health information or not.

3. Method according to claim 1, wherein, if the protected health information in the first data item is not to be de-identified, the method further comprises:
generating a third data item by modifying the first data item by deleting the PHI category information.

4. Method according to claim 1, wherein the first data item comprises one or more tags, the one or more tags comprising the first PHI category information.

5. Method according to claim 1, wherein assessing whether the protected health information in the first data item is to be de-identified or not comprises assessing whether one or more de-identification requirements are fulfilled or not, the one or more de-identification requirements comprising a category requirement, wherein the category requirement is the requirement that the first PHI category is different from one or more given PHI categories of the plurality of PHI categories.

6. Method according to claim 5, further comprising:
accessing de-identification data, the de-identification data comprising information indicative of the one or more de-identification requirements.

7. Method according to claim 6, wherein accessing de-identification data comprises accessing a configuration file, the configuration file comprising the de-identification data.

8. Method according to claim 1, further comprising:
generating a second data file,
wherein, if the protected health information in the first data item is to be de-identified, the second data file comprises the second data item.

9. Method according to claim 1, further comprising:
accessing file information indicative of a file category,
wherein assessing whether the PHI in the first data item is to be de-identified or not is based on the file information.

10. Computer implemented method for generating a first data item, wherein the method comprises:
obtaining protected health information, the protected health information belonging to a first PHI category of a plurality of PHI categories;
determining the first PHI category; and
generating, based on the first PHI category, the first data item, the first data item comprising the protected health information and category information, the category information being indicative of the first PHI category, wherein the first data item is part of a first data file.

11. Method according to claim 10, further comprising:
assessing whether the protected health information meets at least one criteria for protected health information.

12. Method according to claim 1, wherein the first data file is a log file of a laboratory instrument.

13. Method according to claim 1, wherein the first PHI category specifies that the protected health information refers to a biological sample, to patient data, to doctor data or to laboratory test data.

14. A computing device comprising a processor configured to perform the method of claim 1.

15. A laboratory instrument comprising the computing device of claim 14.

16. A non-transitory computer-readable storage medium comprising instructions which, when executed by a computer, cause the computer to carry out the steps of the method of claim 1.
